(19) United States
(12) Patent Application Publication
(10) Pub. No.: US 2021/0338175 A1
(43) Pub. Date: Nov. 4, 2021

(54) SIGNALING DEVICE FOR CATHETERING REQUIREMENT

(71) Applicant: Volker Perlitz, Aachen (DE)

(72) Inventor: Volker Perlitz, Aachen (DE)

(21) Appl. No.: 17/322,461

(22) Filed: May 17, 2021

Related U.S. Application Data
(63) Continuation-in-part of application No. 16/112,699, filed on Aug. 25, 2018, now abandoned.

(30) Foreign Application Priority Data
Aug. 25, 2017 (EP) 17187957.0

(51) Int. Cl. A61B 5/00; A61B 5/0205

(52) U.S. Cl. CPC A61B 5/7275; A61B 5/0205; A61B 5/0004; A61B 5/746; A61B 5/742; A61B 5/7405; A61B 5/6898; A61B 5/7455

(57) ABSTRACT

A signaling device including a data processing device; sensors for heart rate, breathing activity, galvanic skin response, skin blood flow, and movement of a person which respectively include radio communication devices for a wireless connection to the data processing device, wherein the data processing device is configured to wirelessly receive data captured by the sensors regarding a physiological condition of the person and to generate an acoustic, visual or tactile signal as a function of a change of the physiological condition, wherein the data processing device is configured to store the captured data in measurement series, to analyze the measurement series and to detect an increasing or excursive change of the condition in the measurement series, wherein the signal indicates a catheterization requirement of the person.

### RELATED APPLICATIONS

This application is a continuation-in-part of U.S. patent application Ser. No. 16/112,699 filed Aug. 25, 2018, which claims priority from European Patent Application 17 187 957.0 filed on Aug. 25, 2017. The entire teachings of the above applications are incorporated herein by reference.

### FIELD OF THE INVENTION

The invention relates to a signaling device for a catheterization requirement.

### BACKGROUND OF THE INVENTION

Pain from high bladder pressure is often not directly perceived as pain by persons with neurological conditions (e.g., paraplegia, multiple sclerosis or hemiplegia). In healthy and in neurologically impaired individuals alike pain triggers autonomic and behavioral reactions which are commonly termed stress reactions. Such autonomic reactions due to pain-stress stimulus in the wake of high bladder pressure may affect blood pressure, heart rate, breathing frequency, skin blood circulation, and electric skin conductivity. However, in neurological impaired individuals such physiological autonomic reactions may be impeded, aggravated, attenuated or altogether suspended. Autonomic and motoric reactions frequently result in individual autonomic and motoric response patterns with great variation. While e.g. pain-stress reactions may regularly exhibit a leaping decline in heart rate (bradycardia) in a healthy individual, the same pain-stress reaction may fail to trigger bradycardia in a spinal cord injured individual but produces a slowly progressing or a leaping increase of the heart rate, the rate of respiration, electric skin conductivity, and motor responses of the entire body, entire limbs or part of these or any data derived from analysis of primary data instead.

Therefore, synchronous recording of time windows of appropriate lengths of the various signals performed under non-pain-stress reactions and pain-stress reactions are appropriate to establish a correlation basis needed to discriminate individual response patterns from non-pain states.

In addition to medical therapies such as treatment with anticholinergic drugs and/or surgical measures like dissecting the musculus sphincter vesical externus (Reynard J M, Vass J, Sullivan M E, Mamas M: Sphincterotomy and the treatment of detrusor-sphincter dyssynergia: current status, future prospects. Spinal Cord, 2003,41,1-11, doi:10.1038/sj.sc 3101378), intermittent catheterization is used to prevent excessive filling of the bladder. This will empty the bladder through a bladder catheter in approximately even intervals of approximately 6 hours.

Since neither urine production nor bladder capacity nor perception of the filling level of the bladder are constant, those intervals of 6 hours can be too short or too long. For example, infections or psychophysical stress may reduce bladder capacity. In this case, the 6 hours intervals are too short which will result an overservicing as the bladder is not yet filled sufficiently. Overservicing has to be prevented because an invasive process such as insertion of a catheter always bears the risk of bacterial introduction with infection or an injury of the urinary tract (Frankel H L et al.: Long-term survival in spinal cord injury: a fifty year investigation. Spinal Cord 1988; 36: 868-869). An underservicing may occur in case the urinary bladder is filled too much. Chronic underservicing can cause secondary organ damages even including kidney failure.

Reliable assessment of the filling level of the bladder for detecting an individual catheterization requirement before high bladder pressure strikes is conventionally performed non-invasively using sonographic or impedance volumetric measurements (Schlebusch T: lmpedanz-Zystovolumetrie. Aachen, Tech. Hochsch., Diss., 2015).

### BRIEF SUMMARY OF THE INVENTION

Thus, it is an objective of the Invention to determine a suitable point in time for catheterization prior to developing excessive bladder pressure caused by excessive filling of the bladder.

According to the invention, a signaling device is proposed for a catheterization requirement, the signaling device including a data processing device and sensors for recording signal patterns of heart rate, breathing activity, skin blood circulation, electric skin conductivity, and movement of a person, which respectively include radio transmission devices for wireless connection with the data processing device wherein the data processing device is configured to receive data of the sensors regarding a physiological condition of the person wirelessly and to generate an acoustic, visual and/or tactile signal for an increasing (progressive) and/or excursive change of the usual resting condition patterns. The signaling device according to the invention is configured to detect the catheterization requirement of a urinary bladder of a person following analysis of recorded signals able to signal a need for catheterization by discriminating the individual configuration of these signal patterns in states of rest and in states other than pain-stress, such as increased mental or physical load from pain-stress stimuli.

A screenless microcomputer is suitable in particular as a data processing device, typically including a proprietary voltage supply wherein the microcomputer is attached at a bed or a wheelchair, or clothes or can be carried in a bag. Alternatively, also a smartphone of the person is suitable as a data processing device.

In order to measure the heart rate in particular conventional ECG sensors are suitable. For measuring breathing sensor mats or motion sensors are used. For measuring skin blood circulation conventional photoplethysmographic probes can be used. For measuring electric skin conductivity conventional adhesive sensors for electro dermal activity (galvanic skin response, GSR) can be used. For measuring involuntary movements of a body and/or of limbs affected by the neurological conditions, motion sensors can be used.

The radio connection of each sensor to the data processing device facilitates on the one hand side a free positioning of the sensors at the person or to the person's clothes, on the other hand side failure prone or bothersome cable connections are avoided and eventually the possibilities that are provided as a standard in microcomputers and mobile devices for radio connections, in particular Bluetooth, DECT and NFC can be used.

In the signal device according to the invention, the data processing device can be configured particularly through a software or midware (also known as middleware) to wirelessly receive sensor data regarding conditions of the person and to generate an acoustic or tactile signal for an increasing (progressive) and/or leaping change of condition. The software or midware facilitates an adaptation of the data processing device to individually different or changing requirements and sensor configurations, but also in case of a malfunction, maintenance and repair, particularly by updating the software or midware.

Detecting an increasing (progressive) and/or leaping change causes at least the preliminary storage of measurement values and knowledge of a time differential from the next measurement values.

High bladder pressure may cause autonomic pain-stress reactions. Non-autonomic pain-stress reactions are somato-motoric movements and subjective conscious feelings of unease and unrest. The invention is based on observations that the autonomic and somato-motoric pain-stress reactions under high bladder pressure may vary individually with each patient but are always connected to an increasing (progressive) and/or rapid change (increase or decrease) of heart rate, breathing activity, skin blood circulation, involuntary movements, skin humidity or a combination of these features and which are furthermore essentially identical for each patient when repeated. The signaling device according to the invention facilitates an individual detection of the most important autonomic and behavioral states needed to discriminate autonomic pain-stress reactions from non-stress states and thus generates the basis of a reliable signaling of a catheterization requirement when a high bladder pressure occurs.

Advantageously, a signaling device according to the invention includes adjustment devices for manually adjusting sensitivity of the sensors. The signaling device facilitates an adaptation to unconscious individually different pronounced features of the autonomic and somato-motoric pain-stress reaction associated with high bladder pressure.

Advantageously, a signaling device according to the invention includes a control device for manually validating the signal. Thus, the signaling device facilitates a simple documentation and based thereon an adjustment of a threshold for detecting the unconscious increasing (progressive) and/or leaping condition changes of autonomic and somato-motoric pain-stress reaction associated with high bladder pressure.

For adjustment and control devices on the one hand side mechanical twist and slide controls and keying devices can be used which can be configured in particular at the data processing device. Alternatively, virtual keys of such elements can be represented on a screen which is integrated into the data processing device and connected therewith, advantageously via radio, e.g., integrated in a smartphone.

Advantageously, a signaling device of this type according to the invention includes an expert system for automatically calibrating a threshold value for the increasing (progressive) and/or leaping change based on manual validation of signals through the control device. Thus, the signaling device is a system which is continuously learning and self-adjusting its reference basis of the threshold used for detecting the increasing (progressive) and/or leaping condition changes associated with unconscious autonomic and somato-motoric pain-stress reaction associated with high bladder pressure.

Advantageously, a signaling device according to the invention includes additional sensors also for skin blood circulation and skin humidity, for the heart rate, breathing activity and/or for the electrical activity of the brain of the person. The signaling device then includes further information regarding the condition of the person which can be relevant for detecting the increasing (progressive) and/or leaping condition changes associated with unconscious pain-stress reactions associated with high bladder pressure.

In order to measure skin blood circulation and arterial oxygen saturation, sensors for reflection or transmission photo plethysmography (PPG, also pulse oximetry), or nearinfrared spectroscopy (NIRS) are used. For measuring skin humidity sensors for electro dermal activity (galvanic skin response, GSR) are used. For measuring the electric brain activity conventional EEG-sensors, or NIRS are used.

Advantageously, a signaling device according to the invention includes a real-time clock. In the signal device the real time clock does not only provide a cyclic timing that is usable for determining a time differential from the preceding measurements but additionally also provides the option to document the measurements with absolute timestamps.

Advantageously, a signaling device according to the invention includes a signaling element for generating the signal wherein the signaling element is wirelessly connected to the data processing device, e.g., integrated in a smartphone. Alternatively, the signaling device according to the invention can access a signaling element that is integrated in the data processing device. In particular vibrating elements, illuminants, as well as screens and speakers can be used as the signaling element.

Advantageously, a signaling device according to the invention includes a control element that is connected to the data processing device. The data processing device of the signaling device can be configured without control elements and can be arranged at a location that is safe from unauthorized or unintentional access and from other external influences. The control element is advantageously wirelessly connected to the data processing device. Alternatively, a cable-based solution is also suitable.

Advantageously, the control element in a signaling device according to the invention is implemented in a smartphone. For example, the control element can be a software application or a website that is provided by the data processing device wherein the website is called up in a browser of the smartphone. Alternatively, the control element can also be implemented as a software application or a website on a personal computer (PC) that is only temporarily connected to the data processing device by cable. Alternatively, the control element can also be a server application on a server of the manufacturer of the signaling device wherein the data processing device connects automatically with the server or upon request through a GSM-module or through WLAN.

Advantageously, a method for detecting a catheterization requirement according to the invention is implemented by providing a data processing device, and providing sensors that detect two or more of heart rate, breathing activity, galvanic skin response, skin blood flow, and movement of a person which respectively include radio communication devices for a wireless connection to the data processing device. The data processing device is configured to wirelessly receive data captured by the sensors regarding a physiological condition of the person and to generate an acoustic, visual or tactile signal as a function of a change of the physiological condition. The data processing device is configured to store the data in measurement series, to analyze the measurement series and to detect an increasing or excursive change of the physiological condition in the measurement series. The signal indicates a catheterization requirement of the person. Optionally, the number of sensors used may be reduced by limiting the sensors to a subset of sensors able to detect the increasing or excursive change of the physiological condition when at least one the sensors is unable to detect the increasing or excursive change of the physiological condition.

### BRIEF DESCRIPTION OF THE DRAWING

The invention will be described in detail with reference to the following drawings in which like reference numerals refer to like elements wherein:

FIG. 1 illustrates an exemplary embodiment of the signaling device of the invention;

FIG. 2 is a graph of breathing associated chest wall movements with a steady state;

FIG. 3 is a graph of breathing associated chest wall movements with leaping changes;

FIG. 4 is a graph of ECG derived heart rate variability;

FIG. 5 is a graph of ECG derived time series of a heart rate in a resting state;

FIG. 6 is a graph of breathing associated chest wall movements in a healthy volunteer in steady state;

FIG. 7 is a graph of ECG derived heart rate variability with leaping changes;

FIG. 8 is a graph of ECG derived time series of heart rate in a healthy volunteer with an application of a pain stress showing an increasing change; and

FIG. 9 is a graph of ECG derived time series of heart rate in a healthy volunteer with an application of a pain stress showing an excursive change.

### DETAILED DESCRIPTION OF THE INVENTION

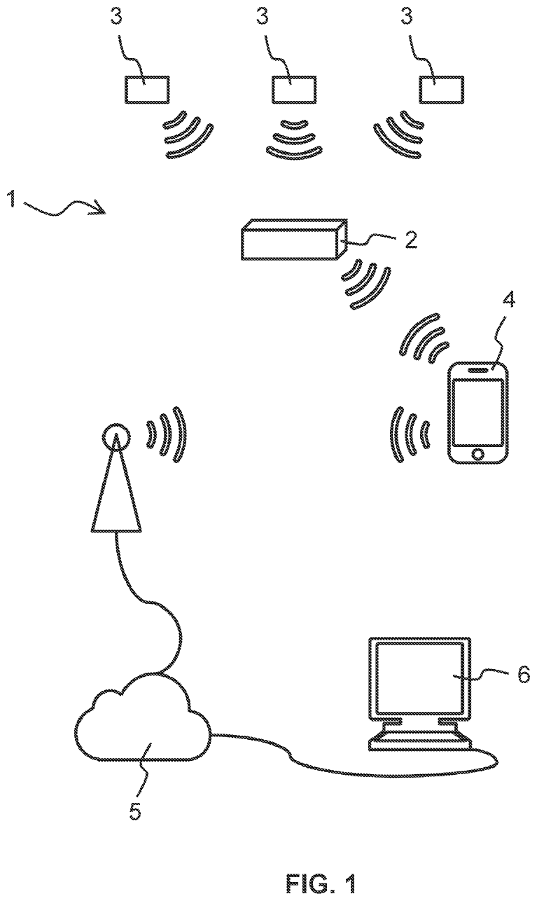

The signaling device 1 illustrated in FIG. 1 includes a data processing device 2, plural (e.g., three or four or more) sensors 3, and a user application that is installed on a smartphone 4 but not illustrated in more detail.

The sensors 3 are configured as a sensor mat for measuring electrical heart activity (ECG, e.g. ADS1292R, Texas Instruments) and breathing of a person that is not illustrated, a photo plethysmography sensor for measuring skin blood circulation combined with a GSR sensor for measuring skin humidity (e. g. Osram SFH7060), and a motion sensor for measuring voluntary and involuntary movements (e. g. ADXL354C, Analog Devices). The sensors 3 respectively include a Bluetooth radio module. The sensor mat is positioned on the upper back area or the chest region. The reflection photo pletysmography sensor is positioned at the ear lobe or incorporated in the sensor mat used to measure electrical heart activity and/or breathing motions. The motion sensor is positioned individually different on predilection locations on the limbs (e.g., a paraplegic patient with involuntary rocking of right big toe indicating catheterization requirement) known to exhibit motion changes due to pain-stress reactions. The GSR sensor is positioned on predilection locations (e.g., sweating inside of the elbow indicating catheterization requirement) known to respond with skin humidity changes to pain-stress reactions.

The reason for such rather unpredictable physical signs indicating catheterization requirement is nerves conduct impulses in the body even when paralyzed. Although the physical signs resulting from the conducted impulses are unpredictable from person to person, the physical signs will be consistent for a given person. Determining such a physical sign and/or predilection location may be achieved by attaching sensors 3 and analyzing the resultant data. There will be one or more differences between the data of a person when a catheterization requirement is indicated (pain-stress reaction) and when a catheterization is not indicated (resting state). The one or more differences will be an increasing (progressive) and/or leaping and/or excursive condition change associated with unconscious autonomic and somato-motoric pain-stress reactions resulting from high bladder pressure.

Initially, a full range of sensors are used but once it is determined which of the sensors are able to detect the increasing or excursive change of the physiological condition, the other sensors may be omitted. Of course, in some instances, there may be other reasons to keep some of the sensors.

The data processing device 2 is a commercially available microcomputer including a processing, an operating memory, a real time clock and a Bluetooth radio module which is configured with a software to wirelessly receive data regarding a condition of a person that is captured by the sensors 3 and to generate a signal for an increasing (progressive) and/or leaping condition change associated with unconscious autonomic and somato-motoric pain-stress reactions associated with high bladder pressure.

In order to use the signaling device 1 the sensors 3 are initially applied to locations at a body of the person which do not further impede the mobility of the typically physically handicapped person. Since unconscious autonomic and somato-motoric pain-stress reactions associated with high bladder pressure may not affect neurologically healthy sections of a neurologically impaired person, save for ECG sensors all other sensors 3 are applied at predilection locations that will supply signals useful for detection of such unconscious autonomic and somato-motoric pain-stress reactions associated with high bladder pressure. Then the data processing device 2 is connected to the smartphone 4 through Bluetooth and configured by the user application.

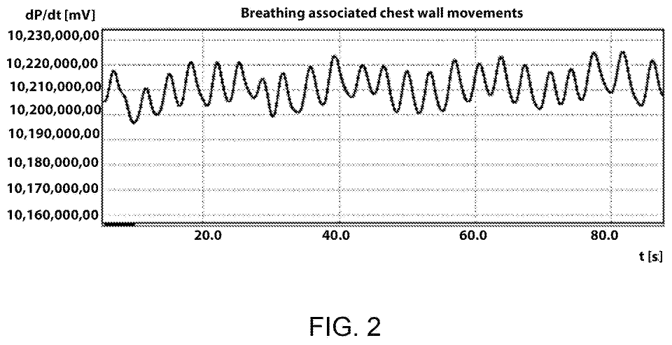

The data processing device 2 detects the sensors 3 arranged in the proximity, establishes a data connection with the sensors via Bluetooth and performs a start configuration of the measurement, namely recording baseline activity at rest. The data processing device 2 thus defines a resting state parameter configuration, divides the distance between two measurements into four time windows and assigns one of the time windows to each of the sensors 3. From this point in time forward, the sensors 3 transmit their respective measuring value regarding the condition of the person in the assigned time window to the data processing device 2 in the measuring rhythm. A typical graphical representation of the values measured is shown in FIGS. 2-9.

The data processing device 2 automatically analyzes the time series of the measurements and detects increasing (progressive) and/or leaping changes associated with unconscious autonomic and somato-motoric pain-stress reactions associated with high bladder pressure indicating alterations of the condition of the person with a sensibility that is initially very high. As soon as the data processing device 2 detects increasing (progressive) and/or leaping changes associated with unconscious autonomic and somato-motoric pain-stress reactions associated with high bladder pressure, the user application generates a tactile, acoustic and visual signal by vibration, a signal tone and an illumination of an LED which may be indicative of a catheterization requirement. Thereupon the user is encouraged to validate the catheterization requirement by actuating a key that is represented on the screen, thus confirming or rejecting the potential catheterization requirement.

With each validated signal an expert system that is implemented in the data processing device 2 learns differentiate which of the increasing (progressive) and/or leaping changes measured indicates an actual catheterization requirement thereby enhancing the discriminatory power of the expert system. A number of erroneous signals decreases approximately exponentially with the number of the signals. Analyzing the measurement series the data processing device 2 also determines the actually required measuring sensitivity of the measuring sensors 3 and the actually required measuring cycle and responds to these analyses with automated adaptation.

Through the user application the user can increase or decrease the measuring sensitivity of the signaling device 1 overall and for each individual sensor any time through virtual slider elements and can lengthen or shorten the measuring cycles.

The user application connects through the internet 5 with a server 6 of the manufacturer in regular intervals when the smartphone 4 provides a data connection and transmits anonymized operating data of the signaling device 1 to the server 6. Based on this data the user application as well as the software of the data processing device 2 is improved continuously.

FIG. 2 is a graph of breathing associated chest wall movements with a steady state.

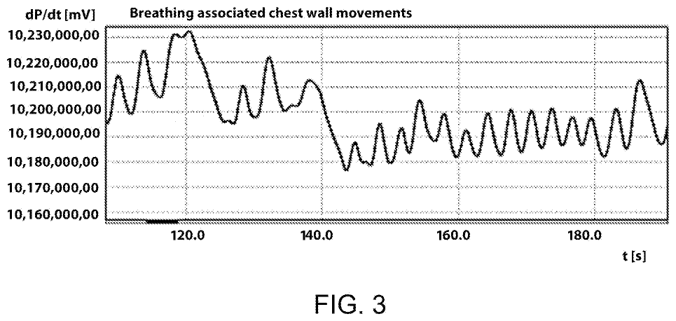

FIG. 3 is a graph of breathing associated chest wall movements with leaping changes.

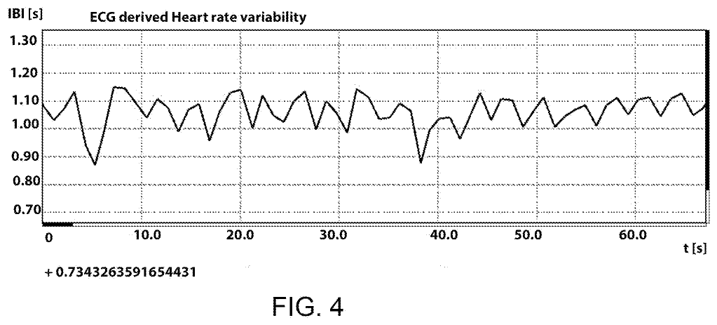

FIG. 4 is a graph of ECG derived heart rate variability.

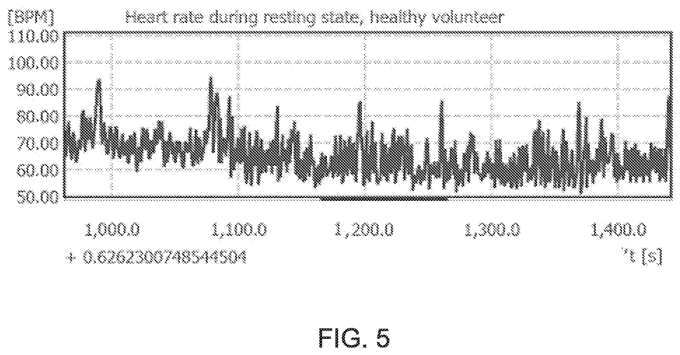

FIG. 5 is a graph of ECG derived time series of a heart rate in a resting state. In FIG. 5, the X-axis is recording time in seconds and the Y-axis is the time between consecutive heart depolarizations converted to beats per minute.

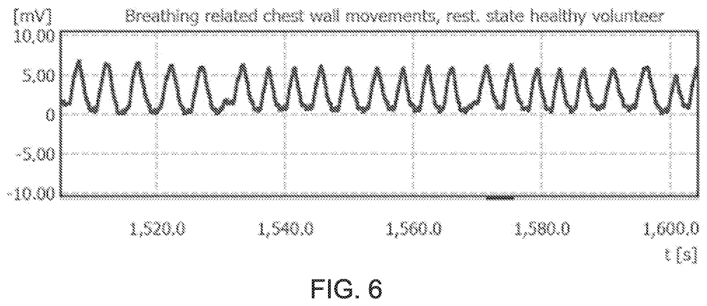

FIG. 6 is a graph of breathing associated chest wall movements in a healthy volunteer in steady state. In FIG. 6, the X-axis is recording time in seconds and the Y-axis is the time between breathing associated thoracic movements in seconds.

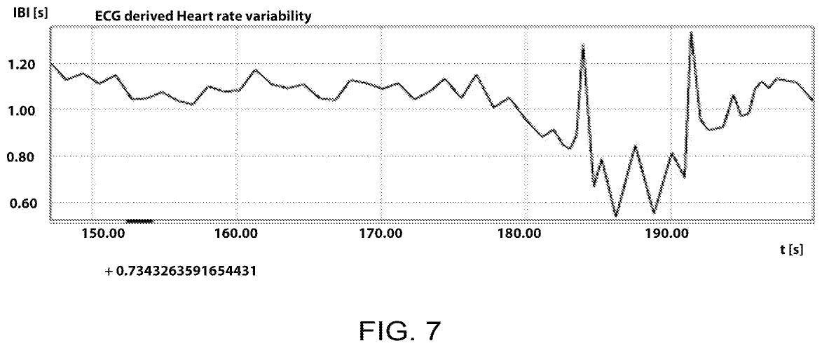

FIG. 7 is a graph of ECG derived heart rate variability with leaping changes.

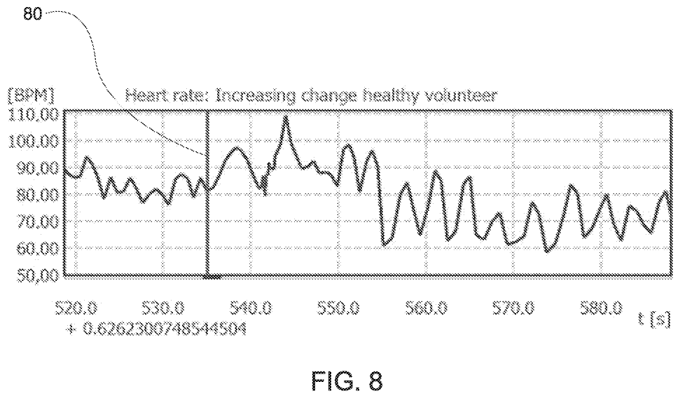

FIG. 8 is a graph of ECG derived time series of heart rate in a healthy volunteer with an application of a pain stress showing an increasing change. The application of pain-stress (cold pressure) occurs at a particular time 80 with a resulting increasing change (negative gain) due to the pain-stress (cold pressure test). The X-axis is recording time in seconds and the Y-axis is the time between consecutive heart depolarizations in seconds.

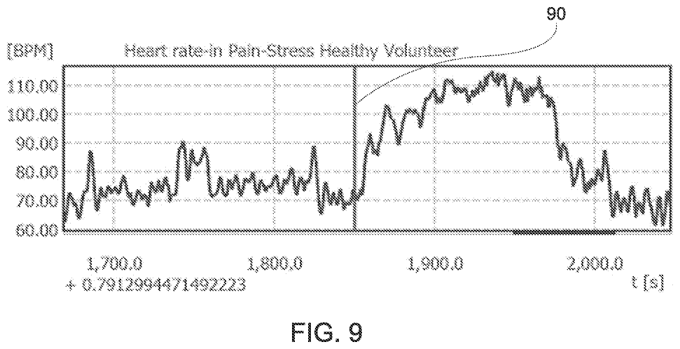

FIG. 9 is a graph of ECG derived time series of heart rate in a healthy volunteer with an application of a pain stress showing an excursive change. The application of pain-stress (cold pressure) occurs at a particular time 90 with a resulting excursive (saltatory) change (positive gain) due to pain-stress (cold pressure test). The X-axis is recording time in seconds and the Y-axis is the time between consecutive heart depolarizations converted to beats per minute.

“Heart rate” (HR) means time intervals between two consecutive depolarizations of the heart muscle per minute measured as electrical activity with unipolar or bipolar ECG. A typical graphical representation of computed HR values is illustrated in FIG. 5.

“Breathing” means chest or abdominal respiration related movements. A typical graphical representation of the values measured is shown in FIG. 2.

“Movement” of a person means changing of posture by positioning limbs or trunk of body or head or all of these or parts thereof. Movement is measured using an accelerometer able to detect changes in at least 3 axes of space.

“Increasing (progressive) change” means continuous positive or negative gain. A typical graphical representation of computed values during experimental pain using cold pressure test (immersing the non-dominant hand in ice cooled water) in a healthy volunteer is illustrated in FIG. 8.

In contrast, an “excursive or leaping change” of the condition means abrupt saltatory change of parameters. A typical graphical representation of an excursive change is illustrated in FIG. 9.

### BRIEF DESCRIPTION OF THE DRAWINGS

1 Signaling device
2 Data processing device
3 Sensor
4 Smartphone
5 Internet
6 Server
80 Particular time
90 Particular time

What is claimed is:

1. A signaling device comprising:
a data processing device; and
sensors that detect one or more of heart rate, breathing activity, galvanic skin response, skin blood flow, and movement of a person which respectively include radio communication devices for a wireless connection to the data processing device,
wherein the data processing device is configured to wirelessly receive data captured by the sensors regarding a physiological condition of the person and to generate an acoustic, visual or tactile signal as a function of a change of the physiological condition,
wherein the data processing device is configured to store the data in measurement series, to analyze the measurement series and to detect an increasing or excursive change of the physiological condition in the measurement series, and
wherein the signal indicates a catheterization requirement of the person.

2. The signaling device according to claim 1, further comprising: adjustment devices for manually adjusting a sensitivity of the sensors.

3. The signaling device according to claim 1, further comprising: a control device for manually validating the signal.

4. The signaling device according to claim 3, further comprising: an expert system configured to automatically calibrate a threshold value for the increasing or excursive change based on a manual validation of the signal by the control device.

5. The signaling device according to claim 1, further comprising: additional sensors that detect skin blood circulation, skin humidity, movements and for heart rate of a person.

6. The signaling device according to claim 1, further comprising: a real time clock.

7. The signaling device according to claim 1, further comprising: a signaling element that is wirelessly connected to the data processing device and configured to generate the signal.

8. The signaling device according to claim 1, further comprising: a control element that is connected to the data processing device.

9. The signaling device according to claim 1, wherein the control element is implemented in a smartphone.

10. The signaling device according to claim 1, wherein the sensors detect the heart rate, the breathing activity, the galvanic skin response, the skin blood flow, and the movement of a person.

11. A method for detecting a catheterization requirement comprising:
providing a data processing device; and
providing sensors that detect two or more of heart rate, breathing activity, galvanic skin response, skin blood flow, and movement of a person which respectively include radio communication devices for a wireless connection to the data processing device,
wherein the data processing device is configured to wirelessly receive data captured by the sensors regarding a physiological condition of the person and to generate an acoustic, visual or tactile signal as a function of a change of the physiological condition,
wherein the data processing device is configured to store the data in measurement series, to analyze the measurement series and to detect an increasing or excursive change of the physiological condition in the measurement series, and
wherein the signal indicates a catheterization requirement of the person.

12. The method of claim 11, further comprising:
limiting the sensors to a subset of sensors able to detect the increasing or excursive change of the physiological condition when at least one of the sensors is unable to detect the increasing or excursive change of the physiological condition.
